(19) United States
(12) Patent Application Publication
(10) Pub. No.: US 2009/0232750 A1
(43) Pub. Date: Sep. 17, 2009

(54) Compositions for indoor tanning

(76) Inventor: John A. St. Cyr, Coon Rapids, MN (US)

Correspondence Address: Kathleen R. Terry, 13840 Johnson Street NE, Ham Lake, MN, 55304, US

(21) Appl. No.: 12/075,624

(22) Filed: Mar. 13, 2008

(51) Int. Cl. A61K 8/60; A61K 31/7004; A61Q 19/04

(52) U.S. Cl. 424/59; 514/23

(57) ABSTRACT

Compositions for inducing a natural tan of the skin is disclosed. The compositions contain D-ribose as the active ingredient plus a penetrant to deliver D-ribose to the basal cells and dermis of the skin. Compositions further containing emollients and emulsifiers are preferred.

### BACKGROUND OF THE INVENTION

The skin is the interface between an organism and the external environment. As such, it is continually subject to stresses such as extreme heat or cold, attack by microorganisms, exposure to UV radiation, abrasion, chemical irritants and the like. As a result, the skin shows signs of damage such as sunburn, roughening, wrinkling, loss of tone, discoloration, and even malignancies, including basal cell carcinoma, squamous cell carcinoma and melanoma. While these effects are often considered to be normal aging, in fact, they are not normal results of aging but are responses to damage.

Many of the effects may be related to free radical formation or suboptimal cell function. The skin is an organ with a high metabolic rate and a high cell turnover. Because it serves as a barrier to microbes, chemicals, radiation, heat and water, skin is highly impermeable. The dermis or corium layer contains cells, blood vessels and nerves. It projects into the epidermis or outer layer in ridges called papillae. The cells of the dermis secrete a ground substance and collagen that give support to the epidermis. The dermis also contains hair follicles and oil glands.

The epidermis is composed of five layers. The basal layer cells are columnar and arranged perpendicularly. These cells divide rapidly to provide a continually renewing epidermis. The basal layer is also the site of collagen and melanin formation. As the layers are pushed up towards the surface of the skin by the formation of new cells, they become progressively more dehydrated and keratinized to form the stratum spinosum, flattened polyhedral cells with short processes or spines; the stratum granulosa, composed of flattened granular cells; the stratum lucidum, composed of several layers of clear transparent cells in which the nuclei are indistinct or absent, and the stratum corneum or cornified layer. This layer is comprised of flattened, dead, keratinized cells that form a barrier to the external environment. As the final step in the ever-renewing metabolism of the skin, the stratum corneum gradually flakes off. Integrity of the skin requires good function in all layers.

In damaged skin, the dermis may secrete less collagen or the collagen may become cross-linked by free radicals or radiation and lose elasticity, resulting in sagging and wrinkling. UV radiation causes the stimulation of the basal cells to produce protective melanin. While a tanned skin is considered a sign of health, actually, a tan is a response to UV damage.

Finally, some skin may have an epidermis that is unnaturally dry and flaky, possibly because of sub-optimal turnover of the dermal cells. Besides detriments to health in having a less functional barrier to infection, damaged skin is less aesthetically pleasing. Unfortunately, the exact areas that are most exposed to the environment and sustain the most damage are those that the subject presents to the world, that is, the face and hands.

Many products have been marketed to improve skin function and appearance. A popular preparation is a cream that incorporates alpha-hydroxy acids, which appear to function as mild irritants that stimulate the exfoliation of the most external stratum corneum, thereby exposing the less cornified and less dry-appearing layers. Unfortunately, many subjects find that these acids are too irritating and may actually cause a red, rough rash. Anti-inflammatory corticosteroids such as hydrocortisone are often used to soothe irritated skin and eliminate unwanted redness. U.S. Pat. No. 6,242,491 discloses that mammals fed creatine show decreased free radical damage in striatal tissue, which is extrapolated to improve skin condition.

Some efforts have been made to improve the condition of skin by applying nutrients topically, on the theory that these nutrients will penetrate the dermis and speed up the turnover of dermal cells, thus presenting younger, more youthful appearing cells to the surface. An example is U.S. Pat. No. 5,053,230, issued Oct. 1, 1991, in which a nutrient medium previously found to support in vitro culture of human epithelial cells was extrapolated to in vivo use to promote the trophism of the skin and related follicles. This complex mixture contains amino acids, monosaccharides, nucleosides and vitamins. Simpler compositions containing sucrose are on the market.

Indoor tanning preparations are available commercially. These products contain chemicals that color the skin, stimulate melanin production or may actually contain melanin. Tanning additives include dihydroxyacetoone, L-tyrosine, copper compounds, octinoate, octsalate and oxybenzone. Many of these preparations give instant “tanning” but the color tends to be somewhat orange, unlike natural melanin.

The need remains for a composition to be applied topically to stimulate the melanocytes to produce natural melanin without exposure to the sun.

### SUMMARY OF THE INVENTION

D-ribose is administered to a subject as a topical composition capable of carrying the D-ribose to the lower layers of the epidermis. The topical composition comprises D-ribose at a concentration of 3 to 20% w/v plus a penetrant and may be incorporated into a pad, a lotion or a cream. The topical composition is most preferably further combined with an emollient in a pharmaceutically acceptable carrier. A bacteriostatic agent can be added. The composition is sprayed or smoothed onto the areas of the skin to be treated, either directly or applied with a pad. The composition is preferably applied daily, most preferably twice or thrice daily, to skin that is to be treated.

When the effect to be desired is tanning of the skin, D-ribose is added to the composition at a concentration of 3% to 20% w/v, more preferably 3% to 8% w/v and most preferably 6% w/v.

A preservative may be added to a lotion or cream. Common preservatives include formalin, methylchloroisothiazine, methylisothiazolinone, butylated hydroxy toluene, and EDTA.

### DETAILED DESCRIPTION OF THE INVENTION

It is known that ATP plays an essential role in the function of all cells. The level of ATP produced may be too low in respect to the requirements of the cells. ATP molecules are constantly recycled in order to furnish the necessary energy. When skin cells are exposed to different stresses, including pollution, smoking, physical exertion, aging, etc., the cells require more energy than can be recycled. The level of ATP may be decreased, affecting cellular metabolism. It has previously been shown that the oral administration of D-ribose raises ATP levels in skeletal muscle (U.S. Pat. Nos. 6,159,942 and 6,534,480). Equally important as its role in ATP formation, D-ribose is its role in the synthesis of DNA and RNA for cell division and growth and for the formation of cyclic AMP and other cell signals.

The effective agent, D-ribose, is used at a concentration of 3 to 20% w/v, preferably from 3 to 8% w/v. and most preferably 6% w/v. The D-Ribose can be administered as a spray in 10% ethanol in water v/v, but is more preferably combined with a penetrant and incorporated into a lotion or cream. The effective agent is most preferably combined with a penetrant and an emollient in a pharmaceutically acceptable carrier. A bacteriostatic agent can be added. The composition is smoothed onto the areas of the skin to be treated, either directly or applied to a cloth pad. The composition is preferably applied daily to skin that is exposed to the external environment, but more preferably two or three times a day.

The topical compositions of the present invention are formulated as solutions or emulsions in a pharmaceutically acceptable carrier. Such a carrier is based on a solvent which is capable of dissolving the active ingredient and which is known to be non-toxic and non-irritating. Such solvents include water, dilute solutions of 1-10% alcohol in water, propylene glycol, glycerol, and mixtures thereof. Acceptable alcohols include ethanol, propanol and butanol. The composition will contain from about 3 to 20% w/v active ingredient.

Lotions and creams are frequently applied to maintain the surface texture of skin. It would be simple and efficacious to include the active ingredient in any commercially available lotion or cream. This invention provides typical formulations of such a lotion or cream.

Preliminary experiments showed that D-ribose applied directly to a wound caused the surface to become brown, possibly due to the Maillard reaction between D-ribose and the proteins in the wound. However, D-ribose in water applied to the skin showed no effect on tanning. Since the object of this invention is to deliver active ingredient to the basal and dermal cells below the epidermis, when the skin barrier is intact, it has been found necessary to include a penetrant in the solutions, lotions or creams of this invention. Such a penetrant will enhance the delivery of active ingredient by transitory disruption of the cell to cell connections of the skin. Penetrants in general include ionic detergents, non-ionic detergents, fatty acids, organic solvents and oils. Certain constructs such as liposomes and nanoparticles can also be used to transport active ingredient through the stratum corneum barrier. It is well known in the industry to select a penetrant with the desired characteristics, which vary in effect from the intense penetration of the solvent dimethyl sulfoxide (DMSO) which cause the rapid co-transport past the skin barrier of any agent present on the skin, to the milder action of dilute ethanol. Of recent interest are the constructs termed liposomes and nanoparticles, which are molecules that form closed structures that can incorporate an active ingredient. Liposomes and nanoparticles can readily penetrate otherwise impermeable barriers.

### Definitions:

“Penetrants” include any compound or construct that enhances the passage of the effective agent through the epidermis to the basal and dermis layers of the skin. Penetrants include but are not limited to, ethanol, butanol, propanol, myristyl alcohol, lauryl sulfate, DMSO, liposomes and nanoparticles.

“Emollients” include materials used for the prevention or relief of dryness and flaking of the epidermis. Emollients include lanolin, mineral oil, glycerin, high molecular weight alcohols such as cetyl alcohol, vegetal oils, animal fats, and the like. Emollients may also have a penetrant effect.

“Pharmaceutically acceptable carrier” means any combination of solvent and additives that effectively suspend the active ingredient. The invention is not limited to any composition or compositions. If the effective ingredient alone is applied, water plus a penetrant is a pharmaceutically acceptable carrier. If the composition includes emollients, an emulsifier is required to hold the emollient in suspension. Emulsifiers include surface active agents such as polysorbate 20 sodium stearyl lactate, glyceryl dilaurate, sorbitol, isopropyl myristate and sodium lauryl sulfate.

“Preservative” includes any bactericidal or bacteriostatic agent commonly used to present bacterial growth in compositions for topical use. Common preservatives include formalin, methylchloroisothiazine, methylisothiazolinone, butylated hydroxy toluene, EDTA and the like.

“Stabilizers” are included. Chelators stabilize the emulsion. Chelators include EDTA and citrate. Other stabilizers include aluminum stearate, sorbitol, dimethicone, and polyethylene glycol.

“Thickeners” are added to control the consistency of the lotion or creme. The texture of the product, from flowable lotion to semi-solid cream, is readily varied by adding thickeners, which are generally higher molecular weight animal fats and animal fat compounds such as oleates, stearates, glycol stearate, and stearyl acetate. Waxes such as cetyl alcohol can be used.

Collagen and hyaluronic acid may be added.

Applicants have previously shown (U.S. Pat. No. 6,663,859) that ribose stimulates the growth of lymphocytes in cell culture. Based on this information, Applicants believe that the same effect might be shown in cell culture with skin cells. The first skin cell tested in vitro was the fibroblast, the culture of which is well known. The Coriell Institute for medical research publishes a web site with detailed instructions, the teachings of which are incorporated by reference. (Http:/locus.umdnj.edu/cer/faq/fibro.html.) Fibroblast cell cultures were tested to determine an optimal level of ribose as compared to glucose control. As more fully explained in U.S. patent application Ser. No. 11/690,707 (“the '707 application”), published Oct. 4, 2007 under publication number 200702312585, a composition containing 1% D-ribose was shown to reduce face wrinkles in female volunteers, presumably through the stimulation of fibroblasts in the basal layer. No tanning effect was reported. Also shown was the protective effect of ribose on cultured normal human fibroblasts subjected to hypoxic shock.

The following examples are provided to further explain how to make and use the invention, but are not limitations on the scope of the invention.

### EXAMPLE 1

### Effect of D-Ribose on Melanocytes In Vivo

To determine the effect of D-ribose on melanocytes, the dermal cells that produce a tanning color when exposed to sunlight, four concentrations of D-ribose suspended in a commercial lotion were applied with a cotton applicator twice a day to two inch areas of A subject's inside forearm. The test area was not exposed to sun light. The composition of the lotion was: water, glycerin, mineral oil, stearic acid, glycol stearate, stearamide AMP, petrolatum, dimethicone, chamomile, helianthus, sambucas, theobroma, seed butter, glyceryl, stearate, cetyl alcohol, triethanolamine, acetylated lanolin alcohol and cetyl acetate.

The results are shown in Table I.

TABLE I

| D- | | | |
|---|---|---|---|
| ribose | Days to first | Days to | |
| w/v | sign of tanning | maximum tan | Comments |
| 1.0% | No tanning | No tanning | |
| 4% | Four | Six | At six days, the tanned area has spread about |
| | | | two inches beyond the application site, that is, |
| | | | the tanned area is about four inches in |
| | | | diameter. The spread area is not as dark as the |
| | | | center of application. |
| 6% | Four | Six | A little darker than the 4%, the tanned area has |
| | | | spread about two inches beyond the application |
| | | | site, that is, the tanned area is about four inches |
| | | | in diameter as in the 4% application. |
| 10% | Four | Six | The tanned area has spread about three inches |
| | | | beyond application site, that is, the tanned area |
| | | | is now about eight inches in diameter. Color is |
| | | | the same as for the 6%. |

Application was stopped after 11 days. Color began to fade at 14 days in all cases and was gone about two weeks after last application. The color was identical to that resulting from normal sun tanning and reached a medium intensity in this fair-skinned subject. The color spread, presumably because of lateral diffusion of the D-ribose within the dermis, stimulating melanocytes outside the application site. The spread area decreased in intensity of color as the diffused D-ribose decreased in concentration. As noted above in the '707 application, lower amounts of D-ribose do not stimulate melanin formation.

In order to ascertain that the tan color was due to the stimulation of melanocytes and not to a chemical reaction such as the Maillard reaction, the subject applied 4% D-ribose in the above referenced lotion to the back of the forearm. The lotion was applied one day only; two days; three days; and four days. All four areas were lightly tanned by day three. By one week after application, the areas to which lotion was applied for one or two days began fading and were completely faded by ten days. The areas to which was applied for three or four days also began fading at one week after application and were completely faded by ten days after last application.

The subject had areas of vitilago, a scar and freckles. The areas of vitilago and scarring did not tan, while freckles got darker, further evidence that the D-ribose stimulated the production of natural melanin.

In other experiments, D-ribose was tested against Jergen's Dark Tanning cream on the upper front thigh of the subject. Each was applied liberally over a six inch by four inch area twice a day for five days. The “tanning” agent in this cream is unknown. The areas on which the Jergen's was applied tanned in 36 hours, hit maximum darkness in two days and faded in eight days. It was noted that the color did not spread, but formed a sharp edge, in contrast to the 4% D-ribose-containing lotion which spread beyond the area of application. In this test, the D-ribose caused maximum darkness in five days and persisted for two weeks after application was discontinued.

D-ribose was also tested against l'Oreal Sublime Glow as above. The active ingredients in this cream were 7.5% octinoate, octsalate 5.0%, octocrylene 3.0% and oxybenzone 6.17%. The area on which the l'Oreal cream was applied darkened within 24 hours and achieved a darker color than with either the Jergen's product or D-ribose containing cream. This color began fading within two days of last application and was completely faded within a week.

In all experiments, the tan area treated with D-ribose was uniform and not blotchy, while the Jergen's and l'Oreal tanned areas were blotchy, evidently corresponding to uneven application. The evenness of the D-ribose tanned areas can be attributed to the natural production of melanin due to the stimulation by D-ribose penetrating and diffusing to the dermal cells. The more rapid fading of the Jergen's and l'Oreal products suggested that the tan color was the result of staining at the surface, rather than stimulation of dermal cells. This suggestion was reinforced by the observation that these creams also stained cuticles and the underside of finger nails.

### EXAMPLE 2

### Simple Solution of Ribose

D-ribose at a concentration of 5% will be dissolved in water plus 1-10% ethanol. The composition will be provided as a solution, preferably in a spray bottle. In use, a spray will be applied on the areas to be treated, most usually the face and hands area. The treatment will be continuous, as long as tanned skin is desired.

### EXAMPLE 3

### Composition with Penetrant

The skin is a barrier. Preliminary effects to determine the effect on improving skin by the application of ribose were unsuccessful, leading the Applicants to the realization that ribose may not have penetrated to the basal cells and dermis. To get penetration it is advantageous to provide a penetrant to increase the concentration of active ingredient at the dermis and epidermis. A preferred penetrant is ethanol, which will be incorporated into the composition at a concentration of 0.1 to 15%, most preferably 10% weight to volume. D-ribose is incorporated at a concentration of from 2 to 10%, preferably 4%, weight to volume.

### EXAMPLE 4

### Lotions and Creams with Ribose

In actual practice, a composition is more convenient and acceptable for use when the D-ribose is incorporated in a composition than includes other conditioning agents, such as emollients and oils. Lotions and creams for topical application differ only in consistency, the ingredients being essentially the same. A cream may be made according to the composition for a lotion by decreasing the water content, by adding a thickening agent or by replacing lower molecular weight fats with higher molecular weight fats or waxes. Color or a whitening agent, fragrance, vitamins, and the like are often added. U.S. Pat. Nos. 4,939,179 and 4,309,448, the teaching of which are hereby incorporated by reference, disclose compositions that have been accepted in the market place. Three compositions for a lotion and a cream according to this invention are:

COMPOSITION A

| Ingredient | Concentration, % | Primary Function |
|---|---|---|
| D-ribose | 6 | Stimulate melanocytes |
| Mineral Oil | 10 | Emollient |
| Isopropyl myristate | 3 | Emulsifier |
| Polyethylene glycol-40 | 4 | Thickener |
| Sorbotan peroleate | 1 | Emollient, Stabilizer |
| Propylene glycol | 1 | Penetrant |
| Cetyl palmitate | 10 | Emollient, Thickener |
| Lanolin alcohol | 10 | Emollient, Thickener |
| Butylated | 0.2 | Preservative |
| hydroxytoluene | | |
| Aluminum Stearate | 4 | Emollient, Stabilizer |
| Sodium lauryl sulfate | 2 | Emulsifier |

COMPOSITION B

| Ingredient | Concentration, % | Primary Function |
|---|---|---|
| D-ribose | 6 | Stimulate melanocytes |
| Glycerin | 8 | Emollient |
| Cetyl esters | 2 | Emollient, Thickener |
| Lanolin oil | 6 | Emollient |
| Sorbitol | 1 | Emulsifier, Stabilizer |
| Dimethicone | 7.5 | Stabilizer |
| Methylparaben | 4 | Thickener |
| Propylparaben | 4 | Thickener |
| SD-alcohol-40-B | 1 | Penetrant |
| Glyceryl dilaurate | 2 | Emulsifier |
| Butylated hydroxytoluene | 0.2 | Preservative |

COMPOSITION C

| Ingredient | Concentration, % | Primary Function |
|---|---|---|
| D-ribose | 6 | Stimulate melanocytes |
| Glycerin | 4 | Emollient |
| Stearic acid | 4 | Emollient, Thickener |
| Glycol stearate | 2 | Emollient, Penetrant |
| Lecithin | 2 | Emulsifier |
| Corn oil | 10 | Emollient |
| Collagen | 1 | Stabilizer, Nutrient |
| Disoldium EDTA | 0.5 | Emulsifier |
| Titanium dioxide | 0.1 | Colorant |
| Yellow dye 10 | 0.05 | Colorant |
| Sodium stearyl lactate | 3 | Emollient, Emulsifier, |
| | | Penetrant |

In each of these compositions, the ingredients are dissolved or suspended in water.

Those skilled in the art can readily make substitutions and additions to the exemplar compositions disclosed within without departing from the spirit and scope of the appended claims.

I claim:

1. A composition comprising an effective amount of D-ribose and a penetrant dissolved in water, the effective amount being sufficient to stimulate the production of melanin when applied to the skin of a subject.

2. The composition of claim 1 wherein the effective amount of D-ribose is 3% to 20% weight to volume.

3. The composition of claim 1 wherein the effective amount of D-ribose is 3% to 8% weight to volume.

3. The composition of claim 1 wherein the effective amount of D-ribose is 6% weight to volume.

4. The composition of claim 1 wherein the penetrant is an alcohol, a surface active agent or an emollient.

5. A composition comprising an effective amount of D-ribose, a penetrant, an emollient and an emulsifier.

6. The composition of claim 5 wherein the effective amount of D-ribose is 3% to 8% weight to volume, the penetrant is an alcohol, a surface active agent or an emollient.

7. The composition of claim 6 wherein the emollient is a mineral oil, a vegetal oil, an animal fat or an alcohol derivative of a vegetal oil or an animal fat.

8. The composition of claim 7 wherein the animal fat is an oleate or a stearate.

9. The composition of claim 6 wherein the emulsifier is polysorbate 20, sodium lauryl sulfate, sodium stearyl lactate, glyceryl dilaurate, sorbitol and isopropyl myristate.

10. A method to induce natural melanin formation in the skin of a subject comprising the topical application of the compositions of claim 1 or claim 5 daily for at least six days or for as long as tanning of the skin of the subject is desires.
